(19) United States
(12) Patent Application Publication
(10) Pub. No.: US 2021/0337802 A1
(43) Pub. Date: Nov. 4, 2021

(54) COMPOSITION AND PROCESS FOR RECONDITIONING RESPIRATORS AND OTHER PERSONAL PROTECTIVE EQUIPMENT

(71) Applicant: Tygrus, LLC, Troy, MI (US)

(72) Inventor: Lawrence Carlson, Oxford, MI (US)

(21) Appl. No.: 17/246,883

(22) Filed: May 3, 2021

Related U.S. Application Data
(60) Provisional application No. 63/019,420, filed on May 3, 2020.

(51) Int. Cl. A01N 59/02; A01N 25/04

(52) U.S. Cl. CPC A01N 59/02; A01N 25/04

(57) ABSTRACT

A process for sanitizing one or more target medical personal protective equipment units, the method that includes the step of contacting the target medical personal protective equipment unit with a charge solution for a contact interval, the contact interval sufficient to infiltrate surfaces located in the interior of the one or more target medical personal protective equipment units. The charge solution includes a polar solvent and an active compound having the chemical formula:

$$⌊ {H}_{x} {O}_{\frac{( x - 1 )}{2}} ⌋ {Z}_{y}$$

wherein x is an odd integer ≥3;
y is an integer between 1 and 20; and
Z is one of a monoatomic ion from Groups 14 and 17 having a charge value between −1 and −3 or a polyatomic ion having a charge between −1 and −3.

The present invention is a non-provisional utility application that claims priority to U.S. Provisional Patent Application Ser. No. 63/019,420 filed May 3, 2020, currently pending, the specification of which is incorporated herein.

### BACKGROUND

The present disclosure pertains to compositions and processes for cleaning reconditioning respirators and other personal protective equipment such as that employed in medical institutions and settings. More particularly, the present disclosure pertains to compositions and processes for cleaning and reconditioning respirators and other personal protective equipment that includes reduction and/or removal of bacterial and/or virologic contaminants from the device being treated.

In order to protect medical personal from disease and patients from infection, it is standard practice for medical personal, and sometimes patients themselves to practice infection control procedures such as wearing face, masks, eye protection, gloves and protective gowns, etc. In situations where the potential for serious infections is high such as during outbreaks of influenza coronavirus and the like, standard medical practices can recommend that medical personal don respirators. Medical grade respirators generally are apparatus that are worn over the mouth and nose to protect the wearer from inhaling hazardous atmospheres such as airborne microorganisms.

Medical bodies such as the United States CDC recommend the use of surgical masks in procedures where there can be aerosol generation from the wearer is small aerosols can produce a disease to the patient. The CDC also recommends the use of respirator masks with a certified degree equivalent to N95 NIOSH or greater to prevent the wearer form the inhalation of infections particles such as Mycobacterium tuberculosis, Avian influenza, severe acute respiratory syndrome (SARS), pandemic influenza, Ebola and coronaviruses such as COVID-19. The degree of the respirator mask of N95 or greater, which filters 95% of airborne particles, is recommended to protect from bacteria and from viruses.

It is believed that certain N95 respirator masks are prepared by melt blowing processes that form the fine mesh of synthetic polymeric fibers that form the inner layer that filter out infectious fibers which is surrounded by spun-bond fabric such as that used in medical protection suits worn in highly infectious situations.

Preferred use instructions call for protective gear such as respirators and other personal protective gear to be single-use; meaning that the health care worker discards the pieces of protective gear after working with an individual patient in order to minimize the opportunity for cross contamination among patients. It can be understood that this practice, though medically understandable, creates large volumes of solid waste that must be treated as potentially biohazardous material and disposed as such. Thus, it would be desirable to provide a composition and process that could be employed on used respirators and other articles of personal protective equipment used in the medical field to reduce the biohazard contaminant load on these articles. This reduction in bio contaminant load can be important prior to disposal. Sufficient bio contaminant load reduction in the respirator or other personal protective gear can render the device or devices suitable for reuse.

Manufacture of such masks as well as other articles of medical personal protective gear is accomplished by capital intensive equipment through a complex interrelated supply chains which can be overtaxed in times of severe medical emergency. This can lead to localized and/or global shortages of fresh personal protective gear. In such situations, there can be a need to clean and reuse such units where possible. Before such reuse occurs, the respirators and/or other personal protection devices should be sanitized to remove biological contaminants, particularly infectious agents from association with the unit. To date, the difficulties attendant with effectively sanitizing units such as N95 respirators has been so great as to preclude effective reuse of such devices. Thus, it would be desirable to provide a compositions and process for cleaning and sanitizing personal protective equipment such as N95 respirators and the like to permit and facilitate its reuse in times of extreme emergency such as pandemic, natural disaster and the like.

### SUMMARY

A process for sanitizing a target medical personal protective equipment unit that includes the step of contacting one or more of the target medical personal protective equipment unit with a charge solution for a contact interval, the contact interval sufficient to infiltrate all surfaces of the target medical personal protective equipment unit. The charge solution that is employed comprises:
an active compound having the chemical formula:

$$⌊ {H}_{x} {O}_{\frac{( x - 1 )}{2}} ⌋ {Z}_{y}$$

wherein x is an odd integer ≥3;
y is an integer between 1 and 20; and
Z is one of a monoatomic ion from Groups 14 and 17 having a charge value between −1 and −3 or a polyatomic ion having a charge between −1 and −3; and
a polar solvent, wherein the charge solution is present as at least one of following: a spray, a vapor, an immersible liquid.

Also disclosed is a process for sanitizing a target medical personal protective equipment unit that includes the step of contacting one or more of the target medical personal protective equipment unit with a charge solution for a contact interval, the contact interval sufficient to infiltrate all surfaces of the target medical personal protective equipment unit. The composition can comprise a material produced by the process that includes the steps of contacting a volume of a concentrated inorganic acid in liquid form having a molarity of at least 7, a density between 22° and 70° baume and a specific gravity between 1.18 and 1.93 in a reaction vessel with an inorganic hydroxide present in a volume sufficient to produce a solid material present in the resulting composition as at least one of a precipitate, a suspended solid, a colloidal suspension; and removing the solid material from the resulting liquid material, wherein the resulting material is a viscous material having a molarity of 200 to 150 M. The therapeutic material also includes water. The therapeutic material can have a pH less than 7, in certain embodiments, less than 5, and in certain embodiments, less than 3.

In certain embodiments the target medical personal protective equipment unit in a face mask or a respirator. In certain embodiments, the face mask or respirator can be a single-use respirator typically covering the mouth and face of the user and configured to filter biological material such as bacteria and viruses.

### BRIEF DESCRIPTION OF THE DRAWINGS

The various features, advantages and other uses of the present apparatus will become more apparent by referring to the following detailed description and drawing in which:

FIG. 1 mass spectra collected in the positive ionization mode for Dilute Sulfuric Acid w/ 400 ppm CaSO₄ (A), Dilute Sulfuric Acid (B), an embodiment as disclosed herein prepared according to the process outlined in Example I (C), and Reverse Osmosis Water (D);

FIG. 2 are mass spectra collected in the negative ionization mode for Dilute Sulfuric Acid w/ 400 ppm CaSO₄ (A), Dilute Sulfuric Acid (B), and embodiment as disclosed herein prepared according to the process outlined in Example I (C), and Reverse Osmosis Water (D).

### DETAILED DESCRIPTION

Disclosed herein is a process for sanitizing one or more target medical personal protective equipment units that comprises the steps of contacting the one or more target personal protective equipment units with a charge solution for an interval sufficient to infiltrate surfaces located in the interior of the one or more target medical personal protective equipment units. The charge solution employed in the contacting step comprises the following:

an active compound having the chemical formula:

$$⌊ {H}_{x} {O}_{\frac{( x - 1 )}{2}} ⌋ {Z}_{y}$$

wherein x is an odd integer ≥3;
y is an integer between 1 and 20; and
Z is one of a monoatomic ion from Groups 14 and 17 having a charge value between −1 and −3 or a polyatomic ion having a charge between −1 and −3; and
a solvent.
   In certain embodiments the solvent employed in the process and composition as disclosed herein can be a polar material and can be present as a fluid and can be a liquid, gas or mixtures of the two. It is also within the purview of this disclosure that during the contacting step, liquid solvent can be present as a liquid, a vaporized material, an atomized material as well as mixtures of the foregoing. The polar solvent material employed will be one that is non-reactive with the material(s) employed in the one or more target medical personal protective equipment units.

In certain embodiments, it is contemplated that the polar solvent can include various polar liquid organic materials. In certain embodiments, the polar solvent can include an organic protic material selected from the group consisting of C1 to C6 alcohols, carboxylic acids having six carbon atoms or less, and mixtures thereof. In certain embodiments, the alcohol that is employed can be one or more of methanol, ethanol, isopropyl alcohol, n-butanol in admixture with water. The carboxylic acid that is chosen can be selected from the group consisting of carboxylic acid can be formic acid, acetic acid, propionic acid, butyric acid and mixtures thereof. In embodiments where the polar solvent has a polar organic liquid component, the solvent can have a water-to-polar liquid organic material ratio between 1 to 1 and 100 to 1, with ratios between 2 to 1 and 50 to 1 in certain embodiments, 10 to 1 and 25 to 1 in certain embodiments. It is also with in the purview of this disclosure to employ water as the solvent material.

The term “personal protective equipment unit” (PPE unit) as that term is used herein includes, but is not limited to, face shields, gloves, goggles and glasses, gowns, head covers, masks, respirators, and shoe covers. In certain embodiments, the PPE unit can be configured as woven or non-woven garments such as head covers, shoe covers, gowns and the like. In certain situations, such garments can be configured as loose-fitting isolation gowns composed of various films, woven, non-woven and/or spun bonded polymeric material such as polyethylene, polypropylene and the like. PPE garments can also be configured as a coverall where desired or required. Non-limiting examples of loose-fitting isolation gowns are those commercially available from various sources such as Grainer supply. Non-limiting examples of coveralls include those commercially available from sources such as 3M.

Respirators as that term is employed herein include, but are not limited to, devices designed to cover the mouth and nose of the health care worker and held in place by a suitable attachment mechanism such as elastic bands, head straps and the like. These devices include units referred to as various disposable filtering facepiece respirators. Non-limiting examples of such respirators include N95 Universal Molded Disposable Respirators commercially available from entities such as 3M, Moldex and the like.

It is contemplated that respirator devices that can be sanitized by the process as described herein can include unvalved N95 respirators as well as valved respirators. In medical applications, the respirator structure can be configured to seat against to the face of the wearer in a manner that encourages airflow through the filter media rather than around the edges of the respirator unit. Non-limiting examples of respirators that can be treated using the process as disclosed herein include disposable respirators commercially available from entities such as 3M, Moldex and the like.

Respirators suitable for the reconditioning process and composition as disclosed herein can be those protective devices that are designed to achieve close facial fit when in the use position on the face of a user to cover the mount and nose. It is believed that the term “N95” is an efficiency rating promulgated by the United States National Institute for Occupational Safety and Health (NIOSH) in which the associated respirator blocks at least 95 percent of airborne test particles having a size of 0.3 microns or greater. This designation can be considered to be the functional equivalent of the FFP2 and FFP3 designation employed in the European Union and KN95 employed in the Peoples Republic of China.

Without being bound to any theory, it is believed that disposable respirators particularly suitable for the process disclosed herein may be those which include at least one interior layer of synthetic polymeric fiber mesh layer. The at least one interior layer can be composed of a melt blown polymeric material such as polypropylene or the like. In certain configurations, the material can be a non-woven polymeric material. The fiber mesh layer can be contoured during manufacture to generally correspond to the face and define a cavity in which the nose and mouth can be positioned such that the upper inner surface of the mask can rest on the ridge of the nose of the wearer and the lower inner surface of the mask can contact he chin region of the wearer. In certain configurations, the fiber mesh layer can be configured with one or more bend central bend regions with at least two generally planar regions that can flex inwardly and outwardly to accommodate the nose and mount of the wearer. Non-limiting examples of some configurations include those discussed in U.S. Pat. Nos. 3,971,373, 4,536,440; 4,850,347; 4,856,509 and the like

The at least one fiber mesh layer in the respirator device can be composed of melt blown polypropylene fiber. Where desired or required, the melt blown fiber mesh can be suitably treated or configured to trap or block biological pathogens such as viruses and bacteria. One non-limiting example of such antiviral treatment technology is that disclosed in patents such as discussed in U.S. Pat. No. 5,387,842 to Roth et al.; U.S. Pat. No. 5,401,446 to Tsai et al; U.S. Pat. Nos. 5,403,453, 5,414,324, U.S. Pat. No. 5,456,972 to Roth et al., the disclosures of which are incorporated by reference herein in their entireties.

Where desired, the respirator mask can also include one or more covering layers overlying the at least one mesh layer, the respirator mask can also include one or mechanisms configured to releasably secure the respirator mask to the face of the wearer. In various configurations, the respirator mask is configured with one or more elastic straps that can either attach to the ears of the wearer or stretch around the back of the head of the wearer.

When first developed, respirator mask such as filtering facepiece respirators were considered single use items to be disposed of in a suitable manner. However, during certain situations such as scarcity and increased medical need, reuse may be necessary. To date no method has been developed and approved for decontamination and reuse. However, decontamination and reuse may need to be considered as a crisis capacity strategy to ensure continued availability.

As disclosed herein, filtering facepiece respirators such as N95 masks can be decontaminated and rendered suitable for reuse by a method that comprises the steps of contacting the filtering facepiece respirator with a charge solution for a contact interval that is sufficient to infiltrate the at least one polymeric mesh layer present in the filtering facepiece respirator.

The charge solution employed comprises and active compound having the chemical formula:

$$⌊ {H}_{x} {O}_{\frac{( x - 1 )}{2}} ⌋ {Z}_{y}$$

wherein x is an odd integer ≥3;
y is an integer between 1 and 20; and
Z is one of a monoatomic ion from Groups 14 and 17 having a charge value between −1 and −3 or a polyatomic ion having a charge between −1 and −3.

It that where desired or required, the active compound can be produced by the process that comprises the steps of:
contacting a volume of a concentrated inorganic acid in liquid form having a molarity of at least 7, a density between 22° and 70° baume and a specific gravity between 1.18 and 1.93 in a reaction vessel with an inorganic hydroxide present in a volume sufficient to produce a solid material present in the resulting composition as at least one of a precipitate, a suspended solid, a colloidal suspension; and
removing the solid material from the resulting liquid material, wherein the resulting material is a viscous material having a molarity of 200 to 150 M.

The active compound can be present in a suitable solvent or carrying medium. The carrying medium can be present as an immersible liquid, an atomized spray, a gaseous vapor or a mixture of the foregoing. In certain embodiments, the carrying medium can be composed of a polar medium such as a polar solvent. The suitable polar solvent can be either aqueous, organic or a mixture of aqueous and organic materials. In situations where the polar solvent includes organic components, it is contemplated that the organic component can include at least one of the following: saturated and/or unsaturated short chain alcohols having less than 5 carbon atoms, and/or saturated and unsaturated short chain carboxylic acids having less than 5 carbon atoms. Where the solvent comprises water and organic solvents, it is contemplated that the water to solvent ratio will be between 1:1 and 400:1, water to solvent, respectively. Non-limiting examples of suitable solvents include various materials classified as polar protic solvents such as water, acetic acid, methanol, ethanol, n propanol, isopropanol, n butanol, formic acid and the like. In certain embodiments, the polar solvent can be water.

The active component can be present in an amount sufficient to contact exterior and interior surfaces of the respirator such as the filtering facepiece respirator and reduce or eliminate biocontaminant material associated therewith. In certain embodiments, the active compound can be present in an amount between 0.1% by volume and 35% by volume; between 0.5 vol % and 35 vol %; between 1 vol % and 35 vol %; between 2 vol % and 35 vol %; between 5 vol % and 35 vol %; between 7 vol % and 35 vol %; between 10 vol % and 35 vol %; between 12 vol % and 35 vol %; between 15 vol % and 35 vol %; between 20 vol % and 35 vol %; 0.1% by volume and 35% by volume; between 0.5 vol % and 35 vol %; between 1 vol % and 35 vol %; between 2 vol % and 30 vol %; between 5 vol % and 30 vol %; between 7 vol % and 30 vol %; between 10 vol % and 30 vol %; between 12 vol % and 30 vol %; between 15 vol % and 30 vol %; between 20 vol % and 30 vol %; 0.1% by volume and 25% by volume; between 0.5 vol % and 25 vol %; between 1 vol % and 25 vol %; between 2 vol % and 25 vol %; between 5 vol % and 25 vol %; between 7 vol % and 25 vol %; between 10 vol % and 25 vol %; between 12 vol % and 25 vol %; between 15 vol % and 25 vol %; between 20 vol % and 25 vol %; 0.1% by volume and 20% by volume; between 0.5 vol % and 20 vol %; between 1 vol % and 20 vol %; between 2 vol % and 20 vol %; between 5 vol % and 20 vol %; between 7 vol % and 20 vol %; between 10 vol % and 20 vol %; between 12 vol % and 20 vol %; between 15 vol % and 20 vol %; 0.1% by volume and 15% by volume; between 0.5 vol % and 15 vol %; between 1 vol % and 15 vol %; between 2 vol % and 15 vol %; between 5 vol % and 15 vol %; between 7 vol % and 15 vol %; between 10 vol % and 15 vol %; between 12 vol % and 15 vol %.

The contact solution is effective at killing or inactivating one or more microbiological organisms filtered and captured by the materials captured and entrained in one or more layers of the filtering material in the respirator. In many instances, the microbiological organisms can include one or more airborne pathogens. Non-limiting examples of airborne pathogens that can be filtered by the respirator unit and can be captured on the respirator material include one or more pathogens such as those within the family Paramyxoviridae (such as measles morbillivirus), Herpesviridae (such as varicella-zoster virus); Mycobacteriaceae (such as Mycobacterium tuberculosis); Orthomyxoviridae (such as influenzavirus A, influenzavirus B); Picornavivdae (such as enterovirus, poliovirus, coxsackie A viruses, coxsackie B viruses and the like); Calicivirdae (such as noroviruses); Coronaviridea including the subfamily Orthocoronavirinae (such as beta coronaviruses like SARS-CoV, SARS-CoV-2, MERS-CoV); Adenoviridae and the like. Respirator use can also provide protection against other pathogens including but not limited to Staphylococcaceae (such as Staphyloccoccu aureus like methicillin-resistant Staphylococcus aureus); Enterococcaceae (including vancomycin-resistant enterococci) and the like.

In use situations, the respirator to be regenerated can have a mixture of various pathogens in different concentrations. The process and material as disclosed herein has been found to be effective is killing both gram-negative and gram-positive bacteria as well as the viruses and other pathogens disclosed.

The pathogen load present in a used respirator can be derived from at least two sources: any germs or pathogens by introduced into the mask material by the wearer when the wearer exhales and any germs or pathogens drawn into the mask material from the surrounding ambient environment has the wearer inhales. Without being bound to any theory, it is believed that pathogen load associated with a given used respirator can be unevenly distributed in and on the structures the associated with the respirator. In certain situations, it is believed that the pathogen load present in a used respirator can be divided into three zones as measured cross-sectionally through the respirator: an outwardly oriented surface zone, a central zone and an inwardly oriented zone as viewed when the respirator is in a use position. In certain embodiments, it is believed that the pathogen load resident in the outwardly oriented zone of the used respirator can be characterized by pathogens generally derived from the ambient surroundings while the pathogens located in the inwardly oriented zone will be characterized, in large part, by pathogens derived from the wearer. The central zone can be characterized by a concentration of pathogens derived from one or both of the foregoing sources. Typically, the concentration of pathogens entrained in the central zone is greater than the concentration of pathogens found in either the outwardly facing zone or the inwardly facing zone is lower than that found entrained in the central zone.

The composition and method disclosed herein permits infiltration of charge solution throughout each of the zones of the respirator to be treated in manner that permits contact between the active compound in the charge solution and pathogens entrained in the various zones in the respirator. Without being bound to any theory, it is believed that contact between the active compound present in the charge solution and charge solution and the pathogen(s) associated with the respirator kills pathogenic material. While the method of pathogenic death is not fully known, it is believed that killing can include denaturing the target pathogenic material by denaturing lysing cellular material in the case of bacterial pathogens, denaturing the lipid envelop in the case of viral pathogens, etc. It is theorized that killing or denaturing the pathogen(s) associated with the respirator surfaces renders the entrained pathogens amenable to dissociation entrainment in the respirator and removal in the charge solution.

In the process as disclosed herein, the contact solution can be brought into contact with the respirator for an interval sufficient to infiltrate the interior zones of the respirator and to remain in contact with the respirator material for an interval sufficient to reduce or eliminate pathogen load associated with the associated respirator. In certain embodiments, the contact interval can be between 10 seconds and 10 minutes; between 30 seconds and 10 minutes; between 1 minute and 10 minutes; between 1.5 minutes and 10 minutes; between 2 minutes and 10 minutes; between 3 minutes and 10 minutes; between 4 minutes and 10 minutes; between 5 minutes and 10 minutes; between 6 minutes and 10 minutes; between 7 minutes and 10 minutes; between 8 minutes and 10 minutes; between 9 minutes and 10 minutes; between 10 seconds and 10 minutes; between 10 seconds and 9 minutes; between 30 seconds and 9 minutes; between 1 minute and 9 minutes; between 1.5 minutes and 9 minutes; between 2 minutes and 9 minutes; between 3 minutes and 9 minutes; between 4 minutes and 9 minutes; between 5 minutes and 9 minutes; between 6 minutes and 9 minutes; between 7 minutes and 9 minutes; between 8 minutes and 9 minutes; between 10 seconds and 8 minutes; between 30 seconds and 8 minutes; between 1 minute and 8 minutes; between 1.5 minutes and 8 minutes; between 2 minutes and 8 minutes; between 3 minutes and 8 minutes; between 4 minutes and 8 minutes; between 5 minutes and 8 minutes; between 6 minutes and 8 minutes; between 7 minutes and 8 minutes; between 10 seconds and 7 minutes; between 30 seconds and 7 minutes; between 1 minute and 7 minutes; between 1.5 minutes and 7 minutes; between 2 minutes and 7 minutes; between 3 minutes and 7 minutes; between 4 minutes and 7 minutes; between 5 minutes and 7 minutes; between 6 minutes and 7 minutes; between 10 seconds and 6 minutes; between 30 seconds and 6 minutes; between 1 minute and 6 minutes; between 1.5 minutes and 6 minutes; between 2 minutes and 6 minutes; between 3 minutes and 6 minutes; between 4 minutes and 6 minutes; between 5 minutes and 6 minutes; between 10 seconds and 5 minutes; between 30 seconds and 5 minutes; between 1 minute and 5 minutes; between 1.5 minutes and 5 minutes; between 2 minutes and 5 minutes; between 3 minutes and 5 minutes; between 4 minutes and 5 minutes; between 10 seconds and 4 minutes; between 30 seconds and 4 minutes; between 1 minute and 4 minutes; between 1.5 minutes and 4 minutes; between 2 minutes and 4 minutes; between 3 minutes and 4 minutes; between 10 seconds and 3 minutes; between 30 seconds and 3 minutes; between 45 seconds and 3 minutes; between 1 minute and 3 minutes; between 1.5 minutes and 3 minutes; between 2 minutes and 3 minutes; between 2.5 minutes and 3 minutes.

The contact between the charge solution and the respirator can occur at a standard temperature and pressure in certain applications. It is also contemplated that the charge solution temperature between 10° C. and 300° C. in certain situations. It is also considered with in the purview of the present disclosure that the contacting step can occur at elevated temperatures where desired or required. It is also within the purview of this disclosure that the contact step can occur at an elevated temperature with the elevated temperature limits being ones that are limited by the thermal degradation temperature of one or more materials present in the respirator or other personal protection equipment.

In certain embodiments, the contact between the charge solution and the respirator can occur at a temperature taken at standard pressure between 10° C. and 15° C.; between 10° C. and 20° C.; between 10° C. and 25° C.; between 10° C. and 35° C.; between 10° C. and 40° C.; between 10° C. and 45° C.; between 10° C. and 50° C.; between 10° C. and 55° C.; between 10° C. and 60° C.; between 10° C. and 65° C.; between 10° C. and 70° C.; between 10° C. and 75° C.; between 10° C. and 80° C.; between 10° C. and 85° C.; between 10° C. and 90° C.; between 10° C. and 95° C.; between 10° C. and 100° C.; between 20° C. and 25° C.; between 20° C. and 35° C.; between 20° C. and 40° C.; between 20° C. and 45° C.; between 20° C. and 50° C.; between 20° C. and 55° C.; between 20° C. and 60° C.; between 20° C. and 65° C.; between 20° C. and 70° C.; between 20° C. and 75° C.; between 20° C. and 80° C.; between 20° C. and 85° C.; between 20° C. and 90° C.; between 20° C. and 95° C.; between 20° C. and 100° C.; between 30° C. and 35° C.; between 30° C. and 40° C.; between 30° C. and 45° C.; between 30° C. and 50° C.; between 30° C. and 55° C.; between 30° C. and 60° C.; between 30° C. and 65° C.; between 30° C. and 70° C.; between 30° C. and 75° C.; between 30° C. and 80° C.; between 30° C. and 85° C.; between 30° C. and 90° C.; between 30° C. and 95° C.; between 30° C. and 100° C.; 10° C. and 15° C.; between 40° C. and 45° C.; between 40° C. and 50° C.; between 40° C. and 55° C.; between 40° C. and 60° C.; between 40° C. and 65° C.; between 40° C. and 70° C.; between 40° C. and 75° C.; between 40° C. and 80° C.; between 40° C. and 85° C.; between 40° C. and 90° C.; between 40° C. and 95° C.; between 40° C. and 100° C.; between 50° C. and 55° C.; between 50° C. and 60° C.; between 50° C. and 65° C.; between 50° C. and 70° C.; between 50° C. and 75° C.; between 50° C. and 80° C.; between 50° C. and 85° C.; between 50° C. and 90° C.; between 50° C. and 95° C.; between 50° C. and 100° C.; between 60° C. and 65° C.; between 60° C. and 70° C.; between 60° C. and 75° C.; between 60° C. and 80° C.; between 60° C. and 85° C.; between 60° C. and 90° C.; between 60° C. and 95° C.; between 60° C. and 100° C.; between 70° C. and 75° C.; between 70° C. and 80° C.; between 70° C. and 85° C.; between 70° C. and 90° C.; between 70° C. and 95° C.; between 70° C. and 100° C.

Where elevated temperatures are employed in the contacting step, the temperature elevation can be accomplished by heating the charge solution to a target temperature that is sufficient to provide the desired elevated temperature during contact. Heating of the process fluid can occur by any suitable heat transfer mechanism.

It is also contemplated that one or more contact intervals can occur with a either an elevation or a decrease in material temperature during the contact interval.

Contact can be accomplished by any suitable mechanism. In certain applications, contact can be accomplished by immersion in either a liquid, gaseous or liquid and gaseous medium composed of the charge solution. In certain embodiments, the respirator is immersed in the charge solution by dipping or submersion. It is also contemplated that various spraying misting or other apparatuses can be employed alone in any suitable administration combination.

The process can also at least one additional contact steps if desired or required. In certain embodiments, the process can include sequential contact with multiple charge solution contact steps. In certain embodiments, the process contemplates discrete charge solutions having the same or different concentrations of the active compound disclosed herein alone or in combination with other components. It is also contemplated that the discrete charge solutions can be held at the same or different temperatures if desired or required. It is also contemplated that that the contact interval can be the same or vary among the various charge solutions, if desired or required. It is also contemplated that the two or more charge solution volumes can be maintained in different states if desired or required. The contact interval for each sequential contact step can have the value as disclosed previously.

The process can also include optionally one or more rinsing steps in which the respirator(s) are contacted with a rinse material such as water after contact with the charge solution is complete. Where desired or required, the rinse material can include one or more components to enhance the filtration capacity of the polymeric material in the respirator.

Where the process includes at least one rinse solution contact step, it is contemplated the interval for contact with the rinse solution can be between 10 seconds and 10 minutes; between 30 seconds and 10 minutes; between 1 minute and 10 minutes; between 1.5 minutes and 10 minutes; between 2 minutes and 10 minutes; between 3 minutes and 10 minutes; between 4 minutes and 10 minutes; between 5 minutes and 10 minutes; between 6 minutes and 10 minutes; between 7 minutes and 10 minutes; between 8 minutes and 10 minutes; between 9 minutes and 10 minutes; between 10 seconds and 10 minutes; between 10 seconds and 9 minutes; between 30 seconds and 9 minutes; between 1 minute and 9 minutes; between 1.5 minutes and 9 minutes; between 2 minutes and 9 minutes; between 3 minutes and 9 minutes; between 4 minutes and 9 minutes; between 5 minutes and 9 minutes; between 6 minutes and 9 minutes; between 7 minutes and 9 minutes; between 8 minutes and 9 minutes; between 10 seconds and 8 minutes; between 30 seconds and 8 minutes; between 1 minute and 8 minutes; between 1.5 minutes and 8 minutes; between 2 minutes and 8 minutes; between 3 minutes and 8 minutes; between 4 minutes and 8 minutes; between 5 minutes and 8 minutes; between 6 minutes and 8 minutes; between 7 minutes and 8 minutes; between 10 seconds and 7 minutes; between 30 seconds and 7 minutes; between 1 minute and 7 minutes; between 1.5 minutes and 7 minutes; between 2 minutes and 7 minutes; between 3 minutes and 7 minutes; between 4 minutes and 7 minutes; between 5 minutes and 7 minutes; between 6 minutes and 7 minutes; between 10 seconds and 6 minutes; between 30 seconds and 6 minutes; between 1 minute and 6 minutes; between 1.5 minutes and 6 minutes; between 2 minutes and 6 minutes; between 3 minutes and 6 minutes; between 4 minutes and 6 minutes; between 5 minutes and 6 minutes; between 10 seconds and 5 minutes; between 30 seconds and 5 minutes; between 1 minute and 5 minutes; between 1.5 minutes and 5 minutes; between 2 minutes and 5 minutes; between 3 minutes and 5 minutes; between 4 minutes and 5 minutes; between 10 seconds and 4 minutes; between 30 seconds and 4 minutes; between 1 minute and 4 minutes; between 1.5 minutes and 4 minutes; between 2 minutes and 4 minutes; between 3 minutes and 4 minutes; between 10 seconds and 3 minutes; between 30 seconds and 3 minutes; between 45 seconds and 3 minutes; between 1 minute and 3 minutes; between 1.5 minutes and 3 minutes; between 2 minutes and 3 minutes; between 2.5 minutes and 3 minutes.

Once the charge solution contact interval (and optional rinse solution contact interval) has been completed, the respirator can be removed from contact with the charge solution and subjected to a drying step to remove residual charge solution that may remain in contact with the respirator. In certain embodiments, the drying step contemplates passive air drying at standard pressure and temperature, passive air drying at elevated temperature and standard pressure, passive air drying at standard temperature and reduced pressure. It is also within the purview of this disclosure that the drying step can include subjecting the respirator to a stream of forced air during all or part of the drying step.

After the drying step is completed, the respirator can be subjected to revalidation steps as desired or required and the processed respirator can be packaged as a reconditioned respirator. The resulting reconditioned respirator can will retain the necessary contours to accomplish positioning, and sealable seating on the face of the user and provide filtration characteristics and performance that meet or exceed the standards of the manufacturer and associated certifying agencies. In certain application, this can be meeting the filtration and performance characteristics outlined in NIOSH N95.

It is contemplated that respirator devices can be subjected to the process disclosed herein multiple times without appreciable degradation in material or performance and that airborne pathogens associated with the respirator can be effectively killed and removed. Where desired or required, the process can be employed on multiple respirators in batches or continuous processes.

The active compound as disclosed herein can be broadly construed as an oxonium ion-derived complex. As defined herein “oxonium ion complexes” are generally defined as positive oxygen cations having at least one trivalent oxygen bond. In certain embodiments the oxygen cation will exist in aqueous solution as a population predominantly composed of one, two and three trivalently bonded oxygen cations present as a mixture of the aforesaid cations or as material having only one, two or three trivalently bonded oxygen cations. Non-limiting examples of oxonium ions having trivalent oxygen cations can include at least one of hydronium ions.

It is contemplated that the in certain embodiments the oxygen cation of the compound will exist in the charge solution in a dissociated or partially dissociated state in which a portion of the compound can be present as a population predominantly composed of one, two and three trivalently bonded oxygen anions present as a mixture of the aforesaid anions or as material having only one, two or three trivalently bonded oxygen anions.

When the active as disclosed herein is admixed with a solvent such as an aqueous or organic solvent, the resulting composition is a solution that can be composed of hydronium ions, hydronium ion complexes and mixtures of the same. Suitable cationic materials can also be referred to as hydroxonium ion complexes. The composition of matter and solutions that contain the same may have utility in various applications where low pH values are desirable. The compounds and materials disclosed herein may also have applicability in a variety of situations not limited to certain cleaning and sanitizing applications.

It has been theorized that extreme trace amounts of cationic hydronium may spontaneously form in water from water molecules in the presence of hydrogen ions. Without being bound to any theory, it is believed that naturally occurring hydronium ions are extremely rare. The concentration of naturally occurring hydronium ions in water is estimated to be no more than 1 in 480,000,000. If they occur at all, hydronium ion compounds are extremely unstable. It is also theorized that naturally occurring hydronium ions are unstable transient species with lifespans typically in the range of nanoseconds. Naturally occurring hydronium ion species are reactive and are readily solvated by water and as such these hydronium ions (hydrons) do not exist in a free state.

In contrast, when the compound disclosed herein is introduced into pure water, the stable hydronium material disclosed herein is one that will remain identifiable. It is believed that the stable hydronium material disclosed herein can complex with water molecules to form hydration cages of various geometries, non-limiting examples of which will be described in greater detail subsequently. The stable compound as disclosed herein, when introduced into a polar solvent such as an aqueous solution is stable and can be isolated from the associated solvent as desired or required.

Conventional strong organic and inorganic acids such as those having a pKₐ≥1.74, when added to water, will ionize completely in the aqueous solution. The ions so generated will protonate existing water molecules to form H₃O+ and associate stable clusters. Weaker acids, such as those having a pKₐ<1.74, when added to water, will achieve less than complete ionization in aqueous solution but can have utility in certain applications. Thus, it is contemplated that the acid material employed to produce the stable electrolyte material can be a combination of one or more acids. In certain embodiments, the acid material will include at least one acid having a pKₐ greater than or equal to 1.74 in combination with weaker acids(s).

It has been found, quite unexpectedly, that the stable active compound as defined herein, when added to an aqueous solution, will produce a polar solvent and provide and effective pKₐ which is dependent on the amount of stable hydronium material added to the corresponding solution independent of the hydrogen ion concentration originally present in that solution. The resulting solution can function as a polar solvent and can have an effective pKₐ between 0 and 5 in certain applications when the initial solution pH prior to addition of the stable hydronium material is between 6 and 8.

The active compound can be added to solutions having an initial pH in the alkaline range, for example between 8 and 12 to effectively adjust the pH of the resulting solvent and/or the effective or actual pKₐ of the resulting solution. Addition of the stable electrolyte material as disclosed herein can be added to an alkaline solution without perceivable reactive properties including, but not limited to, exothermicity, oxidation or the like.

The acidity of theoretical hydronium ions existing in water as a result of aqueous auto-dissociation is the implicit standard used to judge the strength of an acid in water. Strong acids are considered better proton donors than the theoretical hydronium ion material otherwise a significant portion of acid would exist in a non-ionized state. As indicated previously, theoretical hydronium ions derived from aqueous auto-dissociation are unstable as a species, random in occurrence and believed to exist, if at all in extreme low concentration in the associated aqueous solution. Generally, hydronium ions in aqueous solution are present in concentrations between less than 1 in 480,000,000 and can be isolated, if at all, from native aqueous solution via solid or liquid phase organosynthesis as monomers attached to a superacid solution in structures such as HF—SbF₅SO₂. Such materials can be isolated only in extremely low concentration and decompose readily upon isolation.

In contrast, the active compound as disclosed herein, can provide a source of concentrated hydronium ions that are long lasting and can be subsequently isolated from solution if desired or required.

In certain embodiments, the compound can have the following chemical formula:

$$⌊ {H}_{x} {O}_{\frac{( x - 1 )}{2}} + {( {H}_{2} O )}_{y} ⌋ Z$$

wherein x is an odd integer between 3-11;

y is an integer between 1 and 10; and

Z is a polyatomic or monoatomic ion.

The polyatomic ion Z can be an ion that is derived from an acid having the ability to donate one or more protons. The associated acid can be one that would have a pKₐ values ≥1.7 at 23° C. The polyatomic ion Z employed can be one having a charge of +2 or greater. Non-limiting examples of such polyatomic ions include sulfate ions, carbonate ions, phosphate ions, oxalate ions, chromate ions, dichromate ions, pyrophosphate ions and mixtures thereof. In certain embodiments, it is contemplated that the polyatomic ion can be derived from mixtures that include polyatomic ions that include ions derived from acids having pKₐ values ≤1.7.

The active compound material as disclosed herein is stable at standard temperature and pressure and can exist as an oily liquid. The active compound material can be added to water or other polar solvent to produce a polar solution that contains an effective concentration of stable hydronium ion that is greater than 1 part per million. In certain embodiments, the stable electrolyte material as disclosed herein can provide an effective concentration of stable hydronium ion material that is greater than between 10 and 100 parts per million when admixed with a suitable aqueous or organic solvent.

Thus, the addition of the stable hydronium electrolyte material as disclosed herein to an aqueous solution having an initial pH between 6 and 8 results in a solution having an effective pKₐ between 0 to 5. It is also to be understood that the pKₐ of the resulting solution can exhibit a value less than zero as when measured by a calomel electrode, specific ion ORP probe. As used herein the term “effective pKₐ” is a measure of the total available hydronium ion concentration present in the resulting solvent. Thus, it is possible that pH and/or associated pKa of a material when measured may have a numeric value represented between −3 and 7.

Typically, the pH of a solution is a measure of its proton concentration or as the inverse proportion of the —OH moiety. It is believed that the stable electrolyte material as disclosed herein, when introduced into a polar solution, facilitates at least partial coordination of hydrogen protons with the hydronium ion electrolyte material and/or its associated lattice or cage. As such, the introduced stable hydronium ion electrolyte material exists in a state that permits selective functionality of the introduced hydrogen associated with the hydrogen ion.

More specifically, the stable electrolyte material as disclosed herein can have the general formula in certain embodiments:

$$⌊ {H}_{x} {O}_{\frac{( x - 1 )}{2}} ⌋ {Z}_{y}$$

x is an odd integer ≥3;
y is an integer between 1 and 20; and
Z is one of a monoatomic ion from Groups 14 through 17 having a charge between −1 and −3 or a poly atomic ion having a charge between −1 and −3.

In the composition of matter as disclosed herein, monatomic constituents that can be employed as Z include Group 17 halides such as fluoride, chloride, iodide and bromide; Group 15 materials such as nitrides and phosphides and Group 16 materials such as oxides and sulfides. Polyatomic constituents include carbonate, hydrogen carbonate, chromate, cyanide, nitride, nitrate, permanganate, phosphate, sulfate, sulfite, chlorite, perchlorate, hydrobromite, bromite, bromate, iodide, hydrogen sulfate, hydrogen sulfite. It is contemplated that the composition of matter can be composed of a single one to the materials listed above or can be a combination of one or more of the compounds listed.

It is also contemplated that, in certain embodiments, x is an integer between 3 and 9, with x being an integer between 3 and 6 in some embodiments.

In certain embodiments, y is an integer between 1 and 10; while in other embodiments y is an integer between 1 and 5.

The composition of matter as disclosed herein can have the following formula, in certain embodiments:

$$⌊ {H}_{x} {O}_{\frac{( x - 1 )}{2}} ⌋ {Z}_{y}$$

x is an odd integer between 3 and 12;
y is an integer between 1 and 20; and
Z is one of a group 14 through 17 monoatomic ion having a charge between −1 and −3 or a poly atomic ion having a charge between −1 and −3 as outlined above, some embodiments having x between 3 and 9 and y being an integer between 1 and 5.

It is contemplated that the composition of matter exists as an isomeric distribution in which the value x is an average distribution of integers greater than 3 favoring integers between 3 and 10.

The composition of matter as disclosed herein can be formed by the addition of a suitable inorganic hydroxide to a suitable inorganic acid. The inorganic acid may have a density between 22° and 70° baume; with specific gravities between about 1.18 and 1.93. In certain embodiments, it is contemplated that the inorganic acid will have a density between 50° and 67° baume; with specific gravities between 1.53 and 1.85. The inorganic acid can be either a monoatomic acid or a polyatomic acid.

The inorganic acid employed can be homogenous or can be a mixture of various acid compounds that fall within the defined parameters. It is also contemplated that the acid may be a mixture that includes one or more acid compounds that fall outside the contemplated parameters but in combination with other materials will provide an average acid composition value in the range specified. The inorganic acid or acids employed can be of any suitable grade or purity. In certain instances, tech grade and/or food grade material can be employed successfully in various applications.

In preparing the active compound material as disclosed herein, the inorganic acid can be contained in any suitable reaction vessel in liquid form at any suitable volume. In various embodiments, it is contemplated that the reaction vessel can be non-reactive beaker of suitable volume. The volume of acid employed can be as small as 50 ml. Larger volumes up to and including 5000 gallons or greater are also considered to be within the purview of this disclosure.

The inorganic acid can be maintained in the reaction vessel at a suitable temperature such as a temperature at or around ambient. It is within the purview of this disclosure to maintain the initial inorganic acid in a range between approximately 23° and about 70° C. However lower temperatures in the range of 15° and about 40° C. can also be employed.

The inorganic acid is agitated by suitable means to impart mechanical energy in a range between approximately 0.5 HP and 3 HP with agitation levels imparting mechanical energy between 1 and 2.5 HP being employed in certain applications of the process. Agitation can be imparted by a variety of suitable mechanical means including, but not limited to, DC servo drive, electric impeller, magnetic stirrer, chemical inductor and the like.

Agitation can commence at an interval immediately prior to hydroxide addition and can continue for an interval during at least a portion of the hydroxide introduction step.

In the process as disclosed herein, the acid material of choice may be a concentrated acid with an average molarity (M) of at least 7 or above. In certain procedures, the average molarity will be at least 10 or above; with an average molarity between 7 and 10 being useful in certain applications. The acid material of choice employed may exist as a pure liquid, a liquid slurry or as an aqueous solution of the dissolved acid in essentially concentrated form.

Suitable acid materials can be either aqueous or non-aqueous materials. Non-limiting examples of suitable acid materials can include one or more of the following: hydrochloric acid, nitric acid, phosphoric acid, chloric acid, perchloric acid, chromic acid, sulfuric acid, permanganic acid, prussic acid, bromic acid, hydrobromic acid, hydrofluoric acid, iodic acid, fluoboric acid, fluosilicic acid, fluotitanic acid.

In certain embodiments, the defined volume of a liquid concentrated strong acid employed can be sulfuric acid having a specific gravity between 55° and 67° baume. This material can be placed in the reaction vessel and mechanically agitated at a temperature between 16° and 70° C.

In certain specific applications of the method disclosed, a measured, defined quantity of suitable hydroxide material can be added to an agitating acid, such as concentrated sulfuric acid, that is present in the non-reactive vessel in a measured, defined amount. The amount of hydroxide that is added will be that sufficient to produce a solid material that is present in the composition as a precipitate and/or a suspended solid or colloidal suspension. The hydroxide material employed can be a water-soluble or partially water-soluble inorganic hydroxide. Partially water-soluble hydroxides employed in the process as disclosed herein will generally be those which exhibit miscibility with the acid material to which they are added. Non-limiting examples of suitable partially water-soluble inorganic hydroxides will be those that exhibit at least 50% miscibility in the associated acid. The inorganic hydroxide can be either anhydrous or hydrated.

Non-limiting examples of water-soluble inorganic hydroxides include water soluble alkali metal hydroxides, alkaline earth metal hydroxides and rare earth hydroxides; either alone or in combination with one another. Other hydroxides are also considered to be within the purview of this disclosure. “Water-solubility” as the term is defined in conjunction with the hydroxide material that will be employed is defined a material exhibiting dissolution characteristics of 75% or greater in water at standard temperature and pressure. The hydroxide that is utilized typically is a liquid material that can be introduced into the acid material. The hydroxide can be introduced as a true solution, a suspension, or a super-saturated slurry. In certain embodiments, it is contemplated that the concentration of the inorganic hydroxide in aqueous solution can be dependent on the concentration of the associated acid to which it is introduced. Non-limiting examples of suitable concentrations for the hydroxide material are hydroxide concentrations greater than 5 to 50% of a 5 mole material.

Suitable hydroxide materials include, but are not limited to, lithium hydroxide, sodium hydroxide, potassium hydroxide, ammonium hydroxide, calcium hydroxide, strontium hydroxide, barium hydroxide, magnesium hydroxide, and/or silver hydroxide. Inorganic hydroxide solutions when employed may have concentration of inorganic hydroxide between 5 and 50% of a 5 mole material, with concentration between 5 and 20% being employed in certain applications. The inorganic hydroxide material, in certain processes, can be calcium hydroxide in a suitable aqueous solution such as is present as slaked lime.

In the process as disclosed, the inorganic hydroxide in liquid or fluid form is introduced into the agitating acid material in one or more metered volumes over a defined interval to provide a defined resonance time. The resonance time in the process as outlined is considered to be the time interval necessary to promote and provide the environment in which the hydronium ion material as disclosed herein develops. The resonance time interval as employed in the process as disclosed herein is typically between 12 and 120 hours with resonance time intervals between 24 and 72 hours and increments therein being utilized in certain applications.

In various applications of the process, the inorganic hydroxide is introduced into the acid at the upper surface of the agitating volume in a plurality of metered volumes. Typically, the total amount of inorganic hydroxide material will be introduced as a plurality of measured portions over the resonance time interval. Front-loaded metered addition being employed in many instances. “Front-loaded metered addition”, as the term is used herein, is taken to mean addition of the total hydroxide volume with a greater portion being added during the initial portion of the resonance time. An initial percentage of the desired resonance time-considered to be between the first 25% and 50% of the total resonance time.

It is to be understood that the proportion of each metered volume that is added can be equal or can vary based on such non-limiting factors as external process conditions, in situ process conditions, specific material characteristics, and the like. It is contemplated that the number of metered volumes can be between 3 and 12. The interval between additions of each metered volume can be between 5 and 60 minutes in certain applications of the process as disclosed. The actual addition interval can be between 60 minutes to five hours in certain applications.

In certain applications of the process, a 100 ml volume of 5% weight per volume of calcium hydroxide material is added to 50 ml of 66° baume concentrated sulfuric acid in 5 metered increments of 2 ml per minute, with or without admixture. Addition of the hydroxide material to the sulfuric acid produces a material having increasing liquid turbidity. Increasing liquid turbidity is indicative of calcium sulfate solids forming as precipitate. The produced calcium sulfate can be removed in a fashion that is coordinated with continued hydroxide addition in order to provide a coordinated concentration of suspended and dissolved solids.

Without being bound to any theory, it is believed that the addition of calcium hydroxide to sulfuric acid in the manner defined herein results in the consumption of the initial hydrogen proton or protons associated with the sulfuric acid resulting in hydrogen proton oxygenation such that the proton in question is not off-gassed as would be generally expected upon hydroxide addition. Instead, the proton or protons are recombined with ionic water molecule components present in the liquid material.

After the suitable resonance time as defined has passed, the resulting material is subjected to a non-bi-polar magnetic field at a value greater than 2000 gauss; with magnetic fields great than 2 million gauss being employed in certain applications. It is contemplated that a magnetic field between 10,000 and 2 million gauss can be employed in certain situations. The magnetic field can be produced by various suitable means. One non-limiting example of a suitable magnetic field generator is found in U.S. Pat. No. 7,122,269 to Wurzburger, the specification of which is incorporated by reference herein.

Solid material generated during the process and present as precipitate or suspended solids can be removed by any suitable means. Such removal means include, but need not be limited to, the following: gravimetric, forced filtration, centrifuge, reverse osmosis and the like.

The stable electrolyte composition of matter as disclosed herein is a shelf-stable viscous liquid that is believed to be stable for at least one year when stored at ambient temperature and between 50 to 75% relative humidity. The stable electrolyte composition of matter can be use neat in various end use applications. The stable electrolyte composition of matter can have a 1.87 to 1.78 molar material that contains 8 to 9% of the total moles of acid protons that are not charged balanced.

The stable electrolyte composition of matter which results from the process as disclosed herein has molarity of 200 to 150 M strength, and 187 to 178 M strength in certain instances, when measured titrimetrically though hydrogen coulometry and via FFTIR spectral analysis. The material has a gravimetric range greater than 1.15; with ranges greater than 1.9 in in certain instances. The material, when analyzed, is shown to yield up to 1300 volumetric times of orthohydrogen per cubic ml versus hydrogen contained in a mole of water.

It is also contemplated that the composition of matter as disclosed can be introduced into a suitable polar solvent and will result in a solution having concentration of hydronium ions greater than 15% by volume. In some applications, the concentration of hydronium ions can be greater than 25% and it is contemplated that the concentration of hydronium ions can be between 15 and 50% by volume.

The suitable polar solvent can be either aqueous, organic or a mixture of aqueous and organic materials. In situations where the polar solvent includes organic components, it is contemplated that the organic component can include at least one of the following: saturated and/or unsaturated short chain alcohols having less than 5 carbon atoms, and/or saturated and unsaturated short chain carboxylic acids having less than 5 carbon atoms. Where the solvent comprises water and organic solvents, it is contemplated that the water to solvent ratio will be between 1:1 and 400:1, water to solvent, respectively. Non-limiting examples of suitable solvents include various materials classified as polar protic solvents such as water, acetic acid, methanol, ethanol, n-propanol, isopropanol, n-butanol, formic acid and the like.

The ion complex that is present in the solvent material resulting from the addition of the composition of matter as defined therein is generally stable and capable of functioning as an oxygen donor in the presence of the environment created to generate the same. The material may have any suitable structure and solvation that is generally stable and capable of functioning as an oxygen donor. Particular embodiments of the resulting solution will include a concentration of the ion as depicted by the following formula:

$$⌊ {H}_{x} {O}_{\frac{( x - 1 )}{2}} ⌋ +$$

wherein x is an odd integer ≥3.

It is contemplated that ionic version of the compound as disclosed herein exists in unique ion complexes that have greater than seven hydrogen atoms in each individual ion complex which are referred to in this disclosure as hydronium ion complexes. As used herein, the term “hydronium ion complex” can be broadly defined as the cluster of molecules that surround the cation HₓOₓ₋₁+ where x is an integer greater than or equal to 3. The hydronium ion complex may include at least four additional hydrogen molecules and a stoichiometric proportion of oxygen molecules complexed thereto as water molecules. Thus, the formulaic representation of non-limiting examples of the hydronium ion complexes that can be employed in the process herein can be depicted by the formula:

$$⌊ {H}_{x} {O}_{\frac{( x - 1 )}{2}} + {( {H}_{2} O )}_{y} ⌋$$

where x is an odd integer of 3 or greater; and
y is an integer from 1 to 20, with y being an integer between 3 and 9 in certain embodiments.

In various embodiments disclosed herein, it is contemplated that at least a portion of the hydronium ion complexes will exist as solvated structures of hydronium ions having the formula:

H₅+xO_{2y}+

wherein x is an integer between 1 and 4; and

y is an integer between 0 and 2.

In such structures, an

$$⌊ {H}_{x} {O}_{\frac{( x - 1 )}{2}} ⌋ +$$

core is protonated by multiple H₂O molecules. It is contemplated that the hydronium complexes present in the composition of matter as disclosed herein can exist as Eigen complex cations, Zundel complex cations or mixtures of the two. The Eigen solvation structure can have the hydronium ion at the center of an H₉O₄+ structure with the hydronium complex being strongly bonded to three neighboring water molecules. The Zundel solvation complex can be an H₅O₂+ complex in which the proton is shared equally by two water molecules. The solvation complexes typically exist in equilibrium between Eigen solvation structure and Zundel solvation structure. Heretofore, the respective solvation structure complexes generally existed in an equilibrium state that favors the Zundel solvation structure.

The present disclosure is based, at least in part, on the unexpected discovery that stable materials can be produced in which hydronium ion exists in an equilibrium state that favors the Eigen complex. The present disclosure is also predicated on the unexpected discovery that increases in the concentration of the Eigen complex in a process stream can provide a class of novel enhanced oxygen-donor oxonium materials.

The process stream as disclosed herein can have an Eigen solvation state to Zundel solvation state ratio between 1.2 to 1 and 15 to 1 in certain embodiments; with ratios between 1.2 to 1 and 5 to 1 in other embodiments.

The novel enhanced oxygen-donor oxonium material as disclosed herein can be generally described as a thermodynamically stable aqueous acid solution that is buffered with an excess of proton ions. In certain embodiments, the excess of protons ions can be in an amount between 10% and 50% excess hydrogen ions as measured by free hydrogen content.

It is contemplated that oxonium complexes employed in the process discussed herein can include other materials employed by various processes. Non-limiting examples of general processes to produce hydrated hydronium ions are discussed in U.S. Pat. No. 5,830,838, the specification of which is incorporated by reference herein.

The composition disclosed herein has the following chemical structure:

$$⌊ {H}_{x} {O}_{\frac{( x - 1 )}{2}} ⌋ +$$

wherein x is an odd integer ≥3;
y is an integer between 1 and 20; and
Z is a polyatomic or monatomic ion.

The polyatomic ion employed can be an ion derived from an acid having the ability to donate one or more protons. The associated acid can be one that would have a pKa values ≥1.7 at 23° C. The ion employed can be one having a charge of +2 or greater. Non-limiting examples of such ions include sulfate, carbonate, phosphate, chromate, dichromate, pyrophosphate and mixtures thereof. In certain embodiments, it is contemplated that the polyatomic ion can be derived from mixtures that include polyatomic ion mixtures that include ions derived from acids having pKa values ≤1.7.

In certain embodiments, the composition of matter can have the following chemical structure:

$$⌊ {H}_{x} {O}_{\frac{( x - 1 )}{2}} + {( {H}_{2} O )}_{y} ⌋ Z$$

wherein x is an odd integer between 3-11;
y is an integer between 1 and 10; and
Z is a polyatomic ion or monoatomic ion.

The polyatomic ion can be derived from an ion derived from an acid having the ability to donate on or more protons. The associated acid can be one that would have a pKₐ values ≥1.7 at 23° C. The ion employed can be one having a charge of +2 or greater. Non-limiting examples of such ions include sulfate, carbonate, phosphate, oxalate, chromate, dichromate, pyrophosphate and mixtures thereof. In certain embodiments, it is contemplated that the polyatomic ion can be derived from mixtures that include polyatomic ion mixtures that include ions derived from acids having pKₐ values ≤1.7.

In certain embodiments, the composition of matter is composed of a stoichiometrically balanced chemical composition of at least one of the following: hydrogen (1+), triaqua-μ3-oxotri sulfate (1:1); hydrogen (1+), triaqua-μ3-oxotri carbonate (1:1), hydrogen (1+), triaqua-μ3-oxotri phosphate, (1:1); hydrogen (1+), triaqua-μ3-oxotri oxalate (1:1); hydrogen (1+), triaqua-μ3-oxotri chromate (1:1) hydrogen (1+), triaqua-μ3-oxotri dichromate (1:1), hydrogen (1+), triaqua-μ3-oxotri pyrophosphate (1:1), and mixtures thereof in admixture with a polar solvent.

In order to better understand the invention disclosed herein, the following examples are presented. The examples are to be considered illustrative and are not to be viewed as limiting the scope of the present disclosure or claimed subject matter.

### Example I

An active compound as disclosed herein is prepared by placing 50 ml of concentrated liquid sulfuric acid having a mass fraction H₂ SO₄ of 98%, an average molarity(M) above 7 and a specific gravity of 66° baume in a non-reactive vessel and maintained at 25° C. with agitation by a magnetic stirrer to impart mechanical energy of 1 HP to the liquid.

Once agitation has commenced, a measured quantity of sodium hydroxide is added to the upper surface of the agitating acid material. The sodium hydroxide material employed is a 20% aqueous solution of 5M calcium hydroxide and is introduced in five metered volumes introduced at a rate of 2 ml per minute over an interval of five hours with to provide a resonance time of 24 hours. The introduction interval for each metered volume is 30 minutes.

Turbidity is produced with addition of calcium hydroxide to the sulfuric acid indicating formation of calcium sulfate solids. The solids are permitted to precipitate periodically during the process and the precipitate removed from contact with the reacting solution.

Upon completion of the 24-hour resonance time, the resulting material is exposed to a non-bi-polar magnetic field of 2400 gauss resulting in the production of observable precipitate and suspended solids for an interval of 2 hours. The resulting material is centrifuged and force filtered to isolate the precipitate and suspended solids.

The process for sanitizing one or more target medical personal protective equipment units can include the step of contacting the target medical personal protective equipment unit with a charge solution for a contact interval, the contact interval sufficient to infiltrate surfaces located in the interior of the one or more target medical personal protective equipment units. In certain embodiments, the process can include one or more addition processing steps as desired or required. Additional processing steps can be pre-contact or post-contact. Non-limiting examples of post-contact processing steps include at least one of a heat processing step, a forced air exposure step, a UV exposure step, an ozonation step.

### Example II

A second embodiment of the liquid material as disclosed herein is prepared by introducing 50 ml units of concentrated liquid sulfuric acid having a mass fraction H₂ SO₄ of 98%, an average molarity (M) above 7 and a specific gravity of 66° baume into a non-reactive vessel and maintaining each at 25° C. with agitation by a magnetic stirrer to impart mechanical energy of 1 HP to the each liquid unit.

Once agitation has commenced, a measured quantity of sodium hydroxide is added to the upper surface of the agitating acid material of each liquids unit. The sodium hydroxide material employed is a 20% aqueous solution of 5M calcium hydroxide and is introduced in five metered volumes introduced at a rate of 2 ml per minute over an interval of five hours with to provide a resonance time of 24 hours. The introduction interval for each metered volume is 30 minutes.

Turbidity is produced with addition of calcium hydroxide to the sulfuric acid indicating formation of calcium sulfate solids. The solids in each unit are permitted to precipitate periodically during the process and the precipitate is removed from contact with the reacting solution.

Upon completion of the 24-hour resonance time, the resulting material is centrifuged and force filtered to isolate the precipitate and suspended solids from the liquid material and respective resulting material units are collected for further use and analysis.

### Example III

The material produced in Example I is separated into individual samples. Some are stored in closed containers at standard temperature and 50% relative humidity to determine shelf-stability.

### Example IV

To further evaluate the materials prepared in Examples I and II, samples of the materials are diluted with deionized water to provide material that contains 1% by volume of the respective material in water. These samples are evaluated against a dilute sulfuric acid solution, a dilute sulfuric acid solution with to which calcium sulfate is added to yield 300 ppm and a dilute sulfuric with 400 ppm calcium sulfate and well as a reverse osmosis water control.

All samples are diluted in an acid matrix for analysis. The testing is completed using a Thermo iCAP 6300 Duo ICP-OES for calcium and sulfur content following EPA method 200.7.

Each test material is initially prepared by simple dilution in a 5% nitric acid matrix. The calibration standards are prepared in the same acid matrix to match the samples. However, this preparation leads to high recoveries for calcium which is believed to be a result of the sulfuric acid present in the samples but not present in the calibration standards. The calibration standards are re-prepared with a small amount of sulfuric acid in order to match the samples, and the analysis repeated in order to provide better QC recoveries that approach 100%.

In order to test for conductivity the samples are each diluted with de-ionized water for analysis. The testing is completed using a Mettler Toledo Seven Excellence Meter with a conductivity probe following EPA method 120.1. Predicted conductivity results are presented in Table I.

TABLE I
Summary of Conductivity Results

| Sample Name | Conductivity, mS/cm |
|---|---|
| Dilute sulfuric acid | 556 |
| Example I Sample | 551 |
| Example II Sample | 552 |
| Reverse Osmosis Water | 3.2 (μS/cm) |
| Dilute Sulfuric Acid w/300 ppm CaSO₄ | 562 |
| Dilute Sulfuric Acid w/400 ppm CaSO₄ | 558 |

In order to evaluate freezing point, the samples are analyzed using a TA Instruments Q100 DSC equipped with an RCS-90 cooling system following USP <891>. Predicted results are presented in Table II.

TABLE II
Summary of Freeze Point Results

| | Melting |
|---|---|
| Sample Name | Temperature, ° C. |
| Dilute sulfuric acid | −8.73 |
| Example I | −9.07 |
| Example II | −9.05 |
| Reverse Osmosis Water | 0.83 |
| Dilute Sulfuric Acid w/400 ppm CaSO₄ | −9.27 |

The density and specific gravity of the samples are determined at 20° C. using an Anton Paar digital density meter following EPA method 830.7300. predicted results are presented in Table III.

TABLE III
Summary of Density and Specific Gravity Results

| | Density | Specific |
|---|---|---|
| Sample Name | g/cm³ | Gravity |
| Dilute sulfuric acid | 1.0384 | 1.0403 |
| Example I | 1.0403 | 1.0422 |
| Reverse Osmosis Water | 0.9982 | 1.0000 |
| Dilute Sulfuric Acid w/400 ppm CaSO₄ | 1.0400 | 1.0418 |

The samples are also titrated for hydrogen ion content with acidity being determined following ASTM D1067—Test Method A to a pH of 8.6. The testing was completed using a Metrohm 826 Titrando equipped with a pH probe. Predicted results are presented in Table IV.

TABLE IV
Summary of Acidity (Titration) Results

| Sample Name | Acidity @ pH 8.6, meq/L |
|---|---|
| Dilute sulfuric acid | 1276.76 |
| Example I | 1307.28 |
| Example II | 1305.00 |
| Reverse Osmosis Water | 0.08 |
| Dilute Sulfuric Acid w/300 ppm CaSO₄ | 1295.68 |
| Dilute Sulfuric Acid w/400 ppm CaSO₄ | 1260.36 |

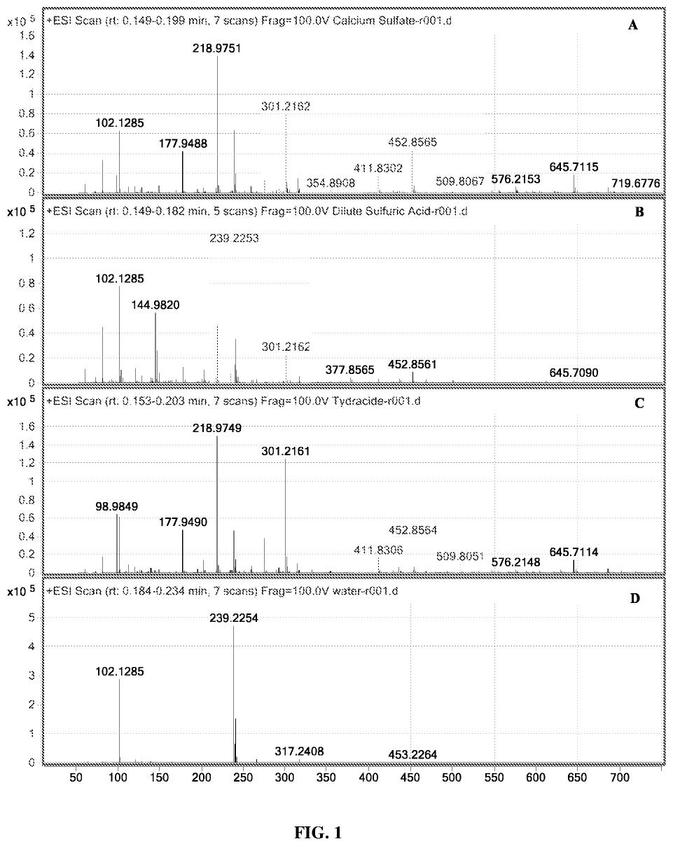

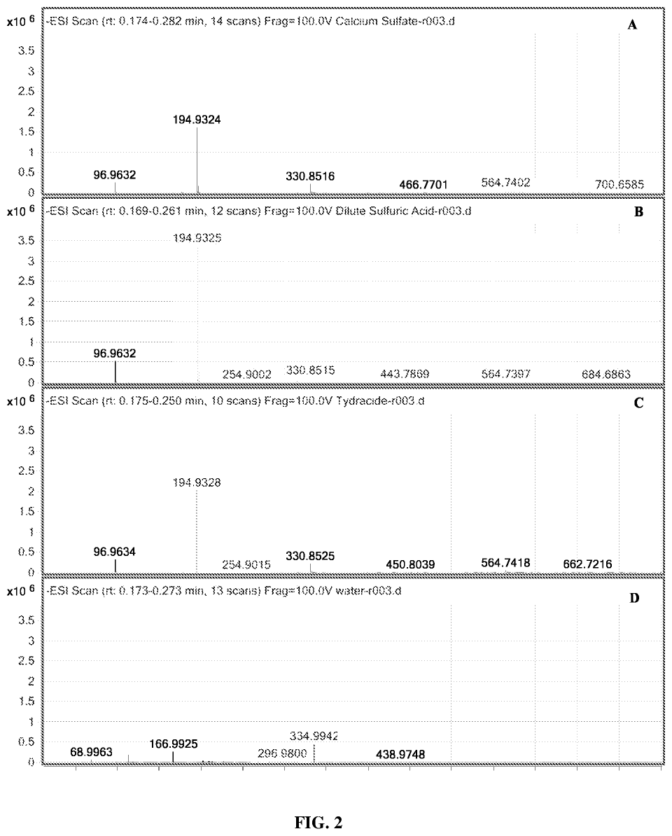

Solutions were analyzed an Agilent 1290/G6530 Q-TOF LC-MS using direct infusion (no column) and electrospray ionization in the positive and negative modes. Representative mass spectra collected in the positive and negative ionization modes are shown in FIGS. 1 and 2 with for Dilute Sulfuric Acid w/ 400 ppm CaSO₄ (A), Dilute Sulfuric Acid (B), Tydracide (C), and Reverse Osmosis Water (D).

Other samples are subjected to analytical procedures to determine composition. The test samples are subjected to FFTIR spectra analysis and titrated with hydrogen coulometry. The sample material has a molarity ranging from 187 to 178 M strength. The material has a gravimetric range greater than 1.15; with ranges greater than 1.9 in in certain instances. The composition is stable and has a 1.87 to 1.78 molar material that contains 8 to 9% of the total moles of acid protons that are not charged balanced. FFTIR analysis indicates that the material has the formula hydrogen (1+), triaqua-μ3-oxotri sulfate (1:1).

### Example V

A 5 ml portion of the material produced according to the method outlined in Example I is admixed in a 5 ml portion of deionized and distilled water at standard temperature and pressure. The excess hydrogen ion concentration is measured as greater than 15% by volume and the pH of the material is determined to be 1.

### Example VI

A composition is prepared in which the active compound of the previous Examples is admixed with distilled water to produce multiple charge solutions that are composed of the active compound in water at concentrations of 1%, 5%, 20% and 25% respectively.

Each charge solution is placed in a large glass beaker and maintained at 1 atm and 70° F.

### Example VII

Personal respirators models 1804 and 1860 commercially available from 3M are obtained and evaluated to determine stability upon exposure to charge solution. One of each model are exposed to a charge solution of a specific concentration by immersing the respirator in the respective charge solution for one minute. Each respirator is weighed prior to immersion and after to confirm that a portion of the charge solution was retained by the respirator.

Each respirator is placed in an exhaust hood for 24 hours and reweighed. Each respirator had a final weight equal to the initial weight indicating that the charge solution had evaporated.

The respirators are visually inspected to assess any structural changes. No alteration is observed. The elastic straps are tested and retain elasticity.

The respirators are tested for blockage and airflow. Airflow through the respirators is not compromised. No degradation of filter performance is observed.

### Example VIII

The cleaning and drying procedure of Example V is repeated over five iterations and the performance of the respirators evaluated. No degradation in performance of structural integrity is observed.

### Example IX

N95 respirator models 1860 and 1804 are set up under laboratory respiratory conditions to simulate eight hours of exposure to specific individual ambient pathogens as outlined in Table I. The exposed respirators are subjected to charge solution containing the active material produced in Example II at concentrations of 1 vol %, 10 vol %, and 20 vol % respectively for an intervals of 1.5 minutes after which the respirators are air dried for an interval of 24 hours. The respective respirators are disassembled and swabbed for pathogens and the tested using ATP testing. No pathogenic infiltration is detected.

TABLE I
Pathogens Under investigation

SARS-CoV-2
staphyloccoccu aureus
mycobacterium tuberculosis
measles morbillivirus

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A process for sanitizing one or more target medical personal protective equipment units, the method comprising the steps of:
contacting the target medical personal protective equipment unit having quantity of at least one microbial pathogen associated therewith, with a charge solution for a contact interval, the contact interval sufficient to infiltrate surfaces located in the interior of the one or more target medical personal protective equipment units, the charge solution comprising:
an active compound having the chemical formula:
$$⌊ {H}_{x} {O}_{\frac{( x - 1 )}{2}} ⌋ {Z}_{y}$$
wherein x is an odd integer ≥3;
y is an integer between 1 and 20; and
Z is one of a monoatomic ion from Groups 14 and 17 having a charge value between −1 and −3 or a polyatomic ion having a charge between −1 and −3; and
a polar solvent, wherein the charge solution is present as at least one of following: a spray, a vapor, an immersible liquid,
wherein after the contact interval, the quantity of the microbiological pathogen is reduced.

2. The process of claim 1 wherein, in the active compound in the charge solution, x is an integer between 3 and 11 and y is an integer between 1 and 10.

3. The process of claim 1 wherein, in the active compound in the charge solution, the polyatomic ion has a charge of −2 or greater.

4. The process of claim 1 wherein, in the active compound in the charge solution, Z is selected from the group consisting of sulfate, carbonate, phosphate, oxalate, chromate, dichromate, pyrophosphate and mixtures thereof.

5. The process of claim 1 wherein the active compound a stiochiometrically balanced chemical composition of at least one of the following: hydrogen (1+), triaqua-μ3-oxotri sulfate (1:1); hydrogen (1+), triaqua-μ3-oxotri carbonate (1:1), hydrogen (1+), triaqua-μ3-oxotri phosphate, (1:1); hydrogen (1+), triaqua-μ3-oxotri oxalate (1:1); hydrogen (1+), triaqua-μ3-oxotri chromate (1:1) hydrogen (1+), triaqua-μ3-oxotri dichromate (1:1), hydrogen (1+), triaqua-μ3-oxotri pyrophosphate (1:1), and mixtures thereof.

6. The process of claim 1 wherein the polar solvent is selected from the group consisting of water, C1-C6 alcohols, carboxylic acids, and mixtures thereof.

7. The process of claim 1 wherein the active compound is present in the polar solvent in an amount between 0.01 and 10 percent by volume.

8. The process of claim 7 wherein the active compound is present in the polar solvent in an amount between 0.1 and 10 percent by volume.

9. The process of claim 8 wherein the active compound is present in the polar solvent in an amount between 0.1 and 2.0 percent by volume.

10. The process of claim 1 wherein the contact solution is maintained at a temperature between 50° C. and 300° C.

11. The process of claim 10 wherein the contact solution is maintained at a temperature between 50° C. and 150° C.

12. The process of claim 1 wherein the microbiological pathogen includes at least one of Mycobacterium tuberculosis, Avian influenza, pandemic influenza, Ebola and coronaviruses.

13. The process of claim 1 wherein the target medical personal protective equipment is a respirator mask.

14. The process of claim 13 wherein the target medical personal protective equipment is an N95 respirator mask.

15. The process of claim 1 further comprising the step of exposing the target medical personal protective equipment unit is subjected to a post contact step, the post contact step including one of exposing the target medical personal protective equipment unit to at least one of a heat processing step, a forced air exposure step, a UV exposure step, an ozonation step.

16. A process for sanitizing one or more target medical personal protective equipment units, the method comprising the steps of:
contacting the target medical personal protective equipment unit with a charge solution for a contact interval, the contact interval sufficient to infiltrate surfaces located in the interior of the one or more target medical personal protective equipment units, the charge solution comprising a stiochiometrically balanced chemical composition of at least one of the following: hydrogen (1+), triaqua-μ3-oxotri sulfate (1:1); hydrogen (1+), triaqua-μ3-oxotri carbonate (1:1), hydrogen (1+), triaqua-μ3-oxotri phosphate, (1:1); hydrogen (1+), triaqua-μ3-oxotri oxalate (1:1); hydrogen (1+), triaqua-μ3-oxotri chromate (1:1) hydrogen (1+), triaqua-μ3-oxotri dichromate (1:1), hydrogen (1+), triaqua-μ3-oxotri pyrophosphate (1:1), and mixtures thereof; and a polar solvent, wherein the charge solution is present as at least one of following: a spray, a vapor, an immersible liquid, wherein the polar solvent is selected from the group consisting of water, C1-C6 alcohols, carboxylic acids, and mixtures thereof, wherein the wherein the active compound is present in the polar solvent in an amount between 0.01 and 10 percent by volume.

17. The process of claim 16 wherein the contact solution is maintained at a temperature between 50° C. and 300° C.

18. The process of claim 16 wherein the target medical personal protective equipment is a respirator mask.

19. The process of claim 16 wherein the microbiological pathogen includes at least one of Mycobacterium tuberculosis, Avian influenza, pandemic influenza, Ebola and coronaviruses.

20. The process of claim 16 wherein the polar solvent is selected from the group consisting of water, C1-C6 alcohols, carboxylic acids, and mixtures thereof.
